(19) United States
(12) Patent Application Publication
(10) Pub. No.: US 2025/0108032 A1
(43) Pub. Date: Apr. 3, 2025

(54) METHODS OF TREATING TYPE 1 DIABETES AND KIDNEY DISEASE

(71) Applicant: LEXICON PHARMACEUTICALS, INC., The Woodlands, TX (US)

(72) Inventor: Michael John Davies, The Woodlands, TX (US)

(73) Assignee: LEXICON PHARMACEUTICALS, INC., The Woodlands, TX (US)

(21) Appl. No.: 18/897,158

(22) Filed: Sep. 26, 2024

Related U.S. Application Data
(60) Provisional application No. 63/541,147, filed on Sep. 28, 2023.

(51) Int. Cl. A61K 31/351; A61K 9/00; A61P 3/10; A61P 13/12

(52) U.S. Cl. CPC A61K 31/351; A61K 9/0053; A61P 3/10; A61P 13/12

(57) ABSTRACT

Methods of using a sodium glucose cotransporter 2 (SGLT2) inhibitor to treat type 1 diabetes, improve cardiovascular health, slow kidney function decline, reduce the risk of cardiovascular death, and reduce the risk of hospitalization for heart failure while minimized adverse events such as diabetic ketoacidosis and severe hypoglycemia are disclosed. In preferred methods, the SGLT2 inhibitor is sotagliflozin. Methods of prescribing and administering sotagliflozin are also disclosed.

This application claims priority to U.S. provisional patent application No. 63/541,147 filed Sep. 28, 2023, the entirety of which is incorporated herein by reference.

### FIELD OF THE INVENTION

This invention relates to methods of treating cardiovascular and renal disease in type 1 diabetic patients while reducing or avoiding adverse effects such as diabetic ketoacidosis.

### BACKGROUND

Despite the broad availability of insulin therapy for people with type 1 diabetes (T1D), there remain large segments of the patient population for whom adequate glycemic control is not consistently achieved. Most adult patients with T1D are under-served by currently available therapies, and most are not able to consistently achieve HbA_{1C} goals on insulin therapy alone.

Adding to these challenges is the fact that people with T1D are at high risk for both kidney and cardiovascular disease. Diabetic kidney disease occurs in approximately 20%-40% of people with T1D and is associated with the development of kidney failure and cardiovascular morbidity and mortality: In fact, cardiovascular disease (CVD) is the main cause of death in type 1 diabetic patients. Stougaard, E. B., et al., “Sotagliflozin, a Dual Sodium-glucose Co-transporter-1 and Sodium-Glucose Co-transporter-2 Inhibitor, Reduces the Risk of Cardiovascular and Kidney Disease, as Assessed by the Steno T1 Risk Engine in Adults with Type 1 Diabetes” Diabetes Obes. Metab. 2023:25:1874-1882.

Sodium glucose cotransporter 2 (SGLT2) inhibitors have proven safe and effective for treating type 2 diabetes and reducing the risk of cardiovascular death, hospitalization for heart failure and urgent heart failure visits. See, e.g., Bhatt, D. L., et al., “Sotagliflozin in Patients with Diabetes and Chronic Kidney Disease” N. Engl. J. Med., 2021 Jan. 14: 384 (2): 129-139. SGLT2 inhibitors have also been shown to be effective in the treatment of kidney disease in patients with and without type 2 diabetes. See, e.g., Heerspink, H. J. L., et al., “Dapagliflozin in Patients with Chronic Kidney Disease” N. Engl. J. Med. 2020:383:1436-46. Unfortunately, the extraordinary benefits conferred by these drugs are generally unavailable to T1D patients.

When administered to T1D patients, SGLT2 inhibitors can improve glycemic control, body weight, blood pressure, and time-in-range with no increased risk of hypoglycemia. Stougaard, supra, 1874. For example, administration of the dual SGLT1/2 inhibitor sotagliflozin “lowered blood pressure and induced mild hemoconcentration, and it was associated with an acute change in eGFR and a reduction in albuminuria” in T1D patients. Van Raalte, D. H., et al., “The Impact of Sotagliflozin on Renal Function, Albuminuria, Blood Pressure, and Hematocrit in Adults With Type 1 Diabetes” Diabetes Care 2019:42:1921-1929, 1927. However, its administration also increased patients' risk of diabetic ketoacidosis (DKA), an excess of ketones in the blood that can cause nausea, pain and malaise, which can require hospitalization and even lead to death. The risk of DKA “is a major limitation in preventing wide-spread uptake and use of SGLT2 inhibitors in patients with T1DM.” Liu, H., et al., “SGLT2 Inhibition in Type 1 Diabetes with Diabetic Kidney Disease: Potential Cardiorenal Benefits Can Outweigh Preventable Risk of Diabetic Ketoacidosis” Current Diabetes Reports, 2022:22:317-332, 327. Indeed, the increased risk of DKA is the reason why SGLT2 inhibitors have not been approved in the United States for use in T1D patients. See, e.g., U.S. Food and Drug Administration, Proposal To Refuse To Approve a New Drug Application for Sotagliflozin Oral Tablets. 200 Milligrams and 400 Milligrams; Opportunity for a Hearing. Federal Register, 2021: 86 (20): 12471-12473, 12472.

### SUMMARY

This invention is directed, in part, to a method of treating type 1 diabetes mellitus (T1D) while minimizing the risk of diabetic ketoacidosis (DKA), which comprises: 1) identifying a patient with type 1 diabetes mellitus and chronic kidney disease (CKD) (i.e., a patient having an estimated glomerular filtration rate (eGFR)<60 mL/min/1.73 m² and/or a urine albumin-to-creatinine ratio (UACR)≥30 mg/g for at least three months); and 2) administering to the patient a therapeutically effective amount of a sodium-glucose cotransporter 2 (SGLT2) inhibitor.

In another embodiment, the invention is directed to a method of improving the cardiorenal health of a patient suffering from type 1 diabetes mellitus while minimizing the risk of DKA, which comprises: 1) identifying a patient with type 1 diabetes mellitus and CKD; and 2) administering to the patient a therapeutically effective amount of an SGLT2 inhibitor. Examples of improvements in cardiorenal health include a lowering of blood glucose, a lowering of systolic blood pressure, a decrease in body weight, increased diuresis/natriuresis, decreased intraglomerular pressure, and decreased proteinuria.

In another embodiment, the invention is directed to a method of slowing kidney function decline in a patient suffering from T1D while minimizing the risk of DKA, which comprises: 1) identifying a patient with type 1 diabetes mellitus and CKD; and 2) administering to the patient a therapeutically effective amount of an SGLT2 inhibitor.

In another embodiment, the invention is directed to a method of improving the safety of an SGLT2 inhibitor administered to patients suffering from T1D, which comprises restricting the administration to T1D patients with CKD.

In another embodiment, the invention is directed to a kit comprising a container (e.g., a box, bottle, blister pack) containing individual dosage forms of an SGLT2 inhibitor, which kit may help achieve any of the various methods described herein. One embodiment encompasses a kit comprising: 1) a container containing at least one solid oral dosage form (e.g., tablet, caplet, or capsule) of an SGLT2 inhibitor; and 2) a label providing prescription information that recommends administration of the SGLT2 inhibitor to patients with type 1 diabetes mellitus who have CKD.

In another embodiment, the invention is directed to a container containing at least one solid oral dosage form of an SGLT2 inhibitor, which container has a label (e.g., printed on or affixed to the outside of the container) providing prescription information that recommends administration of the SGLT2 inhibitor to patients with type 1 diabetes mellitus who have CKD.

### BRIEF DESCRIPTION OF THE DRAWINGS

FIG. 1 shows the cumulative incidence of the first occurrence of cardiorenal events derived from laboratory (eGFR) and non-eGFR data obtained from the SCORED clinical trial.

FIG. 2A shows the placebo-adjusted change in patients' HbA_{1C} for the chronic kidney disease (CKD) and overall cohorts obtained by pooling data from the in Tandem1 and in Tandem2 clinical trials. Results are shown for the oral administration of sotagliflozin (200 mg/day and 400 mg/day) after 24 weeks. P≤0.005 for all comparisons.

FIG. 2B shows the placebo-adjusted change in patients' body weight for CKD and overall cohorts obtained by pooling data from the in Tandem1 and in Tandem2 clinical trials. Results are shown for the oral administration of sotagliflozin (200 mg/day and 400 mg/day) after 24 weeks. P≤0.003 for all comparisons.

FIG. 3A shows the placebo-adjusted change in patients' systolic blood pressure (SBP) for the CKD and overall cohorts obtained by pooling data from the in Tandem1 and in Tandem2 clinical trials. Results are shown for the oral administration of sotagliflozin (200 mg/day and 400 mg/day) after 24 weeks. P≤0.005 for the 400 mg/day data.

FIG. 3B shows the placebo-adjusted percent change in patients' total daily insulin dose for the CKD and overall cohorts obtained by pooling data from the in Tandem1 and in Tandem2 clinical trials. Results are shown for the oral administration of sotagliflozin (200 mg/day and 400 mg/day) after 24 weeks. P≤0.05 for all comparisons.

FIG. 4A shows the placebo-adjusted change in patients' daily bolus insulin dose for the CKD and overall cohorts obtained by pooling data from the in Tandem1 and in Tandem2 clinical trials. Results are shown for the oral administration of sotagliflozin (200 mg/day and 400 mg/day) after 24 weeks. P≤0.005 for the 400 mg/day data. P≤0.001 for the overall comparisons.

FIG. 4B shows the placebo-adjusted percent change in patients' daily basal insulin dose for the CKD and overall cohorts obtained by pooling data from the in Tandem1 and in Tandem2 clinical trials. Results are shown for the oral administration of sotagliflozin (200 mg/day and 400 mg/day) after 24 weeks. P≤0.05 for all comparisons. P≤0.001 for the overall comparisons.

### DETAILED DESCRIPTION

This invention is directed, in part, to methods of using sotagliflozin, which is the generic name for (2S,3R,4R,5S,6R)-2-(4-chloro-3-(4-ethoxy benzyl)phenyl)-6-(methylthio)tetrahydro-2H-pyran-3,4,5-triol:

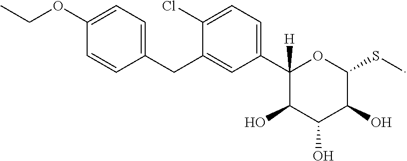

Sotagliflozin is a dual inhibitor of the sodium glucose cotransporters 1 and 2 (SGLT1 and SGLT2), which is sold in the United States under the tradename INPEFA™ to reduce the risk of cardiovascular death, hospitalization for heart failure and urgent heart failure visit in adults with heart failure or adults with type 2 diabetes mellitus, chronic kidney disease and other cardiovascular risk factors. The compound can be prepared by methods disclosed in the art (e.g., U.S. Pat. No. 7,781,577) and formulated as described, for example, in U.S. patent publication no. US20230218650. Crystalline forms of the compound are disclosed in U.S. Pat. No. 8,217,156.

The terms “sodium glucose cotransporter 2 inhibitor” and “SGLT2 inhibitor” are used interchangeably to refer to compounds that inhibit SGLT2. Examples of SGLT2 inhibitors include dapagliflozin, canagliflozin, empagliflozin, sotagliflozin (which also inhibits SGLT1), ipragliflozin, ertugliflozin, tofogliflozin, and luseogliflozin, and pharmaceutically acceptable salts and solvates thereof.

As used herein, the terms “type 1 diabetes”, “T1D”, “type 1 diabetes mellitus” and “T1DM” are used interchangeably.

The term “chronic kidney disease” (CKD) generally refers to abnormalities in a patient's kidney structure or function, present for greater than three months, with implications for health. KDIGO 2012 Clinical Practice Guideline for the Evaluation and Management of Chronic Kidney Disease, Kidney International Supplements, 2013:3 at x. The disease is classified based on cause, glomerular filtration rate (GFR) category and albuminuria (CGA) category. The GFR categories are: G1 (normal or high)≥90 ml/min/1.73 m²; G2 (mildly decreased) 60-89 ml/min/1.73 m²; G3a (mildly to moderately decreased) 45-59 ml/min/1.73 m²; G3b (moderately to severely decreased) 30-44 ml/min/1.73 m²; G4 (severely decreased) 15-29 ml/min/1.73 m²; and G5 (kidney failure)<15 ml/min/1.73 m². The CGA categories are: A1 (normal to mildly increased)<30 mg/g (<3 mg/mmol); A2 (moderately increased) 30-300 mg/g (3-30 mg/mmol); and A3 (severely increased)>300 mg/g (>30 mg/mmol). Id. Both GFR and CGA can be measured by well-established techniques. Estimating equations for GFR (eGFR) based on serum creatinine (SCr), but not SCr alone, are sensitive for detecting GFR<60 mL/min/1.73 m². A decreased eGFR using SCr can be confirmed by GFR estimation using an alternative filtration marker (cystatin C) or GFR measurement, as necessary. Id. at 21.

Unless otherwise indicated, a patient with CKD according to the present invention has an eGFR<60 mL/min/1.73 m² (e.g., <45 mL/min/1.73 m²) and/or a urine albumin-to-creatinine ratio (ACR or UACR)≥30 mg/g (3 mg/mmol) (e.g., >300 mg/g (>30 mg/mmol)) for more than three months. In some embodiments of the invention, the patient has both an eGFR<60 mL/min/1.73 m² and a UACR≥30 mg/g (3 mg/mmol) for more than three months. In certain embodiments, the patient has an eGFR<45 mL/min/1.73 m² and >15 mL/min/1.73 m² (i.e., does not have a GFR category of G5).

This invention encompasses methods of safely treating T1D, improving cardiovascular health, slowing kidney function decline, reducing the risk of cardiovascular death and reducing the risk of hospitalization for heart failure, which comprise administering a therapeutically effective amount of an SGLT2 inhibitor (e.g., sotagliflozin).

Unless otherwise indicated, the terms “safe”, “safely”, “improving the safety”, “minimize the risk” and “minimizing the risk”, when used herein to describe a method of this invention using an SGLT2 inhibitor, refer to a method that reduces the relative risk of DKA and/or hypoglycemia versus placebo as compared to the relative risk observed when the SGLT2 inhibitor is administered to a T1D patient without CKD (e.g., a patient with a GFR category of G1 or G2 and a CGA category of A1).

One embodiment of the invention is directed to a method of treating T1D while minimizing the risk of diabetic ketoacidosis (DKA), which comprises: 1) identifying a patient with T1D and chronic kidney disease (CKD) (i.e., a patient having an estimated glomerular filtration rate (eGFR)<60 mL/min/1.73 m² and/or a urine albumin-to-creatinine ratio (UACR)≥30 mg/g for at least three months); and 2) administering to the patient a therapeutically effective amount of a sodium-glucose cotransporter 2 (SGLT2) inhibitor.

Another embodiment of the invention is directed to a method of improving the cardiorenal health of a patient suffering from T1D while minimizing the risk of DKA, which comprises: 1) identifying a patient with T1D and CKD; and 2) administering to the patient a therapeutically effective amount of an SGLT2 inhibitor. Examples of improvements in cardiorenal health include a lowering of blood glucose, a lowering of systolic blood pressure, a decrease in body weight, increased diuresis/natriuresis, decreased intraglomerular pressure and decreased in proteinuria.

Another embodiment of the invention is directed to a method of slowing kidney function decline in a patient suffering from T1D while minimizing the risk of DKA, which comprises: 1) identifying a patient with T1D and CKD; and 2) administering to the patient a therapeutically effective amount of an SGLT2 inhibitor.

Another embodiment of the invention is directed to a method of reducing the risk of sustained eGFR decline, end stage kidney disease, cardiovascular death or hospitalization for heart failure in a patient at risk thereof while minimizing the risk of DKA, which comprises: 1) identifying a patient with T1D and CKD; and 2) administering to the patient a therapeutically effective amount of an SGLT2 inhibitor.

Another embodiment of the invention encompasses a method of improving the safety of an SGLT2 inhibitor administered to patients suffering from T1D, which comprises restricting the administration to T1D patients with CKD.

In particular embodiments of the invention, the SGLT2 inhibitor is dapagliflozin, canagliflozin, empagliflozin, sotagliflozin, ipragliflozin, ertugliflozin, tofogliflozin, or luscogliflozin, or a pharmaceutically acceptable salt or solvate thereof. A preferred SGLT2 inhibitor is sotagliflozin.

In particular embodiments of the invention, the patient is on optimized insulin therapy. In particular methods of the invention, the patient is on insulin therapy, and the method further comprises optimizing that therapy.

In particular embodiments of the invention, the patient has an eGFR<60 mL/min/1.73 m², an eGFR<45 mL/min/1.73 m², or an eGFR<30 mL/min/1.73 m². In particular embodiments of the invention, the patient has an eGFR>15 mL/min/1.73 m² (i.e., does not have a GFR category of G5).

In particular embodiments of the invention, the patient has a UACR≥30 mg/g. In some, the patient has a UACR≥300 mg/g.

In particular embodiments of the invention, the patient has both an eGFR<60 mL/min/1.73 m² and a UACR≥30 mg/g.

In particular embodiments of the invention, the patient has a BMI≥27 kg/m².

In particular embodiments of the invention, the SGLT2 inhibitor is administered to the patient for more than 52 weeks (e.g., 1.5, 2, 5 or 10 years). In such embodiments, monitoring and/or reporting of DKA events occurs over the entire period during which the drug is administered, preferably in a regular manner (e.g., daily, weekly or monthly).

In particular embodiments of the invention, the patient satisfies at least one of the following criteria: is an adult (i.e., is at least 18 years of age): is not a pregnant woman: is using either multiple daily injections (MDI) or subcutaneous insulin infusion (SCII) for insulin delivery; has an HbA_{1C} of 7.0-11.0%; has a β-hydroxybutyrate (BHB) level≤0.6 mmol/L. In some embodiments, the patient is an adult. In some, the patient satisfies all of these criteria.

In particular embodiments of the invention, the SGLT2 inhibitor (e.g., sotagliflozin) is administered orally. In some, the SGLT2 inhibitor is administered as a tablet, caplet, or capsule. In embodiments comprising the use of sotagliflozin, a therapeutically effective amount of sotagliflozin is at least 200 mg/day. In some embodiments, the therapeutically effective amount of sotagliflozin is at least 400 mg/day.

Particular methods of the invention comprise monitoring the patient for DKA or an increased risk of DKA. This can be achieved by methods known in the art, such as by testing the patient's blood glucose, arterial pH, and/or serum bicarbonate levels. Particular methods of the invention comprise monitoring the patient for hypoglycemia or an increased risk of hypoglycemia, which monitoring can also be done by methods well known in the art.

This invention also encompasses a kit comprising a container (e.g., a box, bottle, blister tabs) containing individual dosage forms of an SGLT2 inhibitor. In one embodiment, the kit comprises: 1) a container containing at least one solid oral dosage form (e.g., tablet, caplet, or capsule) of an SGLT2 inhibitor; and 2) a label providing prescription information that recommends administration of the SGLT2 inhibitor to patients with T1D who have CKD.

In one embodiment, the kit further comprises a means of measuring and/or reporting DKA events to a health care provider. Means of measuring DKA events include ketone test kits and meters (e.g., glucose meters) that measure blood ketone levels. Means of reporting DKA events include information containing email or messaging (e.g., texting) instructions. Other means include mobile apps that may wirelessly connect to glucose meters and store and/or report ketone levels to a health care provider or an agent thereof.

Another embodiment encompasses a container (e.g., a box, bottle, blister tabs) containing at least one solid oral dosage form of an SGLT2 inhibitor, which container has a label (e.g., printed on or affixed to the outside of the container) providing prescription information that recommends administration of the SGLT2 inhibitor to patients with T1D who have CKD.

Kits and containers of the invention may contain any SGLT2 inhibitor, such as dapagliflozin, canagliflozin, empagliflozin, sotagliflozin, ipragliflozin, ertugliflozin, tofogliflozin or luscogliflozin, or a pharmaceutically acceptable salt or solvate thereof. A preferred SGLT2 inhibitor is sotagliflozin (e.g., in a dosage form comprising 200 mg or 400 mg of the drug).

In particular embodiments of the invention, the label recommends restricting the administration to patients having an eGFR>15 mL/min/1.73 m². In some, the label recommends restricting the administration to patients satisfying at least one of the following criteria: is an adult (i.e., is at least 18 years of age): is not a pregnant woman: is using either multiple daily injections (MDI) or subcutaneous insulin infusion (SCII) for insulin delivery; has an HbA_{1C} of 7.0-11.0%; has a β-hydroxybutyrate (BHB) level≤0.6 mmol/L.

### EXAMPLES

### Example 1. Analysis of Cardiorenal Endpoints in Patients with Type 2 Diabetic

The cardiorenal protective properties of sotagliflozin in patients with type 2 diabetes informed research that led to the present invention. Those properties were observed in a Phase 3, multicenter, double-blind clinical trial conducted in patients with type 2 diabetes mellitus (glycated hemoglobin level, ≥7%) and estimated glomerular filtration rate (eGFR) of 25 to 60 ml per minute per 1.73 m² of body-surface area. The primary end point was changed during the trial to the composite of the total number of deaths from cardiovascular causes, hospitalizations for heart failure, and urgent visits for heart failure.

Of 19.188 patients screened, 10,584 were enrolled, with 5,292 assigned to the sotagliflozin group and 5,292 assigned to the placebo group and followed for a median of 16 months. The rate of primary end-point events was 5.6 events per 100 patient-years in the sotagliflozin group and 7.5 events per 100 patient-years in the placebo group: hazard ratio, 0.74; 95% confidence interval (CI), 0.63 to 0.88; P<0.001. The rate of deaths from cardiovascular causes per 100 patient-years was 2.2 with sotagliflozin and 2.4 with placebo (hazard ratio, 0.90; 95% CI, 0.73 to 1.12; P=0.35). For the original coprimary end point of the first occurrence of death from cardiovascular causes, nonfatal myocardial infarction, or nonfatal stroke, the hazard ratio was 0.84 (95% CI, 0.72 to 0.99); for the original coprimary end point of the first occurrence of death from cardiovascular causes or hospitalization for heart failure, the hazard ratio was 0.77 (95% CI, 0.66 to 0.91). Diarrhea, genital mycotic infections and volume depletion were more common with sotagliflozin than with placebo.

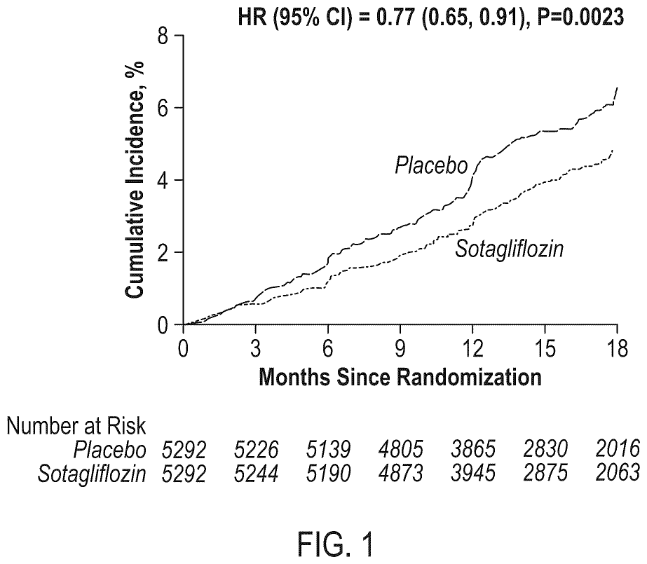

As shown in FIG. 1, sotagliflozin significantly reduced the risk of cardiorenal endpoints as compared to placebo. Table 1 provides the number and type of cardiorenal events observed during the trial, wherein each of the sotagliflozin and placebo groups contained 5292 patients.

TABLE 1
Effect of Sotagliflozin on Cardiorenal Endpoints
in Patients with Type 2 Diabetes and CKD

| Events | Sota n (%) | Placebo n (%) | HR (95% CI) | p-value |
|---|---|---|---|---|
| First event in composite | 239 (4.5) | 306 (5.8) | 0.77 (0.65, 0.91) | 0.0023 |
| 50% decline in eGFR | 75 (1.4) | 121 (2.3) | 0.60 (0.45, 0.81) | 0.0006 |
| (sustained or last value) | | | | |
| End-stage kidney disease | 50 (0.9) | 78 (1.5) | 0.63 (0.45, 0.91) | 0.0122 |
| eGFR <15 mL/min/m² | 41 (0.8) | 68 (1.3) | 0.60 (0.41, 0.88) | 0.0090 |
| (sustained or last value) | | | | |
| Chronic dialysis or | 15 (0.3) | 22 (0.4) | 0.67 (0.35, 4.86) | 0.6618 |
| transplant | | | | |
| Renal death | 8 (0.2) | 5 (0.1) | 1.59 (0.52, 4.86) | 0.6618 |
| CV death | 155 (2.9) | 170 (3.2) | 0.90 (0.73, 1.12) | 0.3626 |

HR, hazard ratio; CI, confidence interval.

This study clearly showed that patients with diabetes and kidney disease with or without albuminuria who took daily sotagliflozin had a lower risk of the composite of deaths from cardiovascular causes, hospitalizations for heart failure and urgent visits for heart failure than placebo. See, e.g., U.S. patent application Ser. No. 17/574,977, filed Jan. 13, 2022; Bhatt, D. L., et al., supra; Sridhar, V. S., et al., “Sotagliflozin and Kidney Outcomes, Kidney Function, and Albuminuria in Type 2 Diabetes and CKD” CJASN 2024:19:557-564. Sotagliflozin has since been approved by the United States Food and Drug Administration (FDA) to reduce the risk of cardiovascular death, hospitalization for heart failure, and urgent heart failure visit in patients with heart failure or type 2 diabetes mellitus, chronic kidney disease, and other cardiovascular risk factors. INPEFA™ Prescribing Information, May 2023.

### Example 2. In Tandem1 and in Tandem2 Studies in Patients with Type 1 Diabetes

The efficacy of sotagliflozin as adjunct therapy in adult patients with type 1 diabetes mellitus who have inadequate glycemic control with insulin therapy was evaluated in two multicenter, double-blind clinical studies named “in Tandem1” and “in Tandem2”. Patients were randomized into three treatment groups: those treated with sotagliflozin 200 mg/day, those treated with sotagliflozin 400 mg/day; and those treated with placebo.

Details of the in Tandem1 study have been reported. See, e.g., Buse, J. B., et al., “Sotagliflozin in Combination With Optimized Insulin Therapy in Adults With Type 1 Diabetes: The North American in Tandem1 Study” Diabetes Care 2018; 41: 1970-1980. Details of the in Tandem2 study have also been reported. See, e.g., Danne. T., et al., “HbA and Hypoglycemia 1c Reductions at 24 and 52 Weeks With Sotagliflozin in Combination With Insulin in Adults With Type 1 Diabetes: The European in Tandem2 Study” Diabetes Care 2018; 41: 1981-1990. Both studies included men and nonpregnant women aged≥18 years with T1D using either multiple daily injections (MDI) or subcutaneous insulin infusion (SCII) for insulin delivery and whose HbA_{1C} was 7.0-11.0% at screening. Patients with β-hydroxybutyrate (BHB) levels>0.6 mmol/L at screening were excluded.

In both studies, insulin therapy was optimized for six weeks prior to randomization, and optimized insulin continued until the study conclusion at week 52. After a two-week placebo run-in, patients were randomly assigned to double-blind treatment with sotagliflozin 200 or 400 mg or placebo for 52 weeks. Insulin optimization, which refers to the adjustment of insulin to meet standard-of-care glycemic targets, starting six weeks prior to randomization and continued for the entire study. An independent insulin dose monitoring committee (IDMC) assessed standard-of-care adherence and provided feedback to the principal investigator if deviations from standard of care were observed prior to week 24. HbA_{1c} was masked to study staff during this period. Between week 24 and 52, insulin optimization continued without input from the IDMC, and HbA1c values were unmasked. Safety was monitored for 30 days after the last dose of study medication.

The primary endpoint of both studies was the change from baseline to week 24 in glycated hemoglobin A1C of either dose of sotagliflozin (400 mg or 200 mg) treatment group compared to placebo. Other endpoints included the change from baseline of sotagliflozin versus placebo in: the proportion of patients with A1C<7.0% and no episode of severe hypoglycemia and no episode of DKA; body weight, bolus insulin dose, fasting plasma glucose (FPG), diabetes treatment satisfaction questionnaire status (DTSQs) score; and Diabetes Distress Screening Scale (DDS2) questionnaire score.

Efficacy analyses were based on the mITT (modified Intent-to-Treat) population. Primary efficacy analyses were conducted based on data collected during the Core Treatment Period (Baseline through and including the week 24 visit assessments). As shown below in Table 2, sotagliflozin achieved its primary endpoint by leading to statistically significant lower A1C levels for both doses when compared to placebo in the pooled in Tandem1/in Tandem2 study results after the core treatment and long-term extension periods.

TABLE 2
Pooled Efficacy and Safety Results from Randomization to Week 52

| | | | |
|---|---|---|---|
| | Placebo | 200 mg/day | 400 mg/day |
| Characteristic | n = 526 | n = 524 | n = 525 |

Baseline (BL) Characteristics

| | | | |
|---|---|---|---|
| Age years, mean (sd) | 42.5 (13.3) | 44.4 (13.7) | 44.0 (13.4) |
| HbA1c percentage, mean (sd) | 7.66 (0.81) | 7.68 (0.77) | 7.64 (0.78) |
| Weight kg, mean (sd) | 84.25 (17.56) | 84.46 (18.13) | 84.23 (18.11) |
| BMI kg/m², mean (sd) | 28.54 (5.28) | 28.89 (5.56) | 28.74 (5.18) |

Efficacy through 24 (primary efficacy time point) and 52 Weeks

| | | | |
|---|---|---|---|
| HbA1c change from Baseline | | | |
| Week 24 Treatment Comparison | N/A | −0.36 | −0.38 |
| LS Mean (SE), % | | | |
| Week 52 Treatment Comparison | N/A | −0.23 | −0.32 |
| LS Mean (SE), % | | | |
| Weight, LS Mean (SE) kg | | | |
| Percent Change (%) from | 0.47 (0.137) | −1.70 (0.137) | −2.55 (0.137) |
| Baseline at Week 24 | | | |
| Treatment Comparison vs. PBO | N/A | −2.17 (0.188) | −3.02 (0.188) |
| Percent Change (%) from | 0.91 (0.209) | −2.31 (0.208) | −3.33 (0.209) |
| Baseline at Week 52 | | | |
| Treatment Comparison vs. PBO | N/A | −3.21 (0.291) | −4.24 (0.291) |
| No Weight Gain at Week 52 | | | |
| (SOTA vs. PBO) | | | |
| Percentage HbA1c <7.0% | 9.1 | 21.8 | 26.1 |
| Absolute difference in %^{a} | N/A | 12.6 | 17.0 |
| % with absolute reduction | 8.8 | 23.7 | 28.8 |
| in HbA1c ≥0.5% | | | |
| Absolute difference in %^{a} | N/A | 14.9 | 20.0 |

Patients with Safety Event through 52 Weeks

| | | | |
|---|---|---|---|
| Any TEAE, n (%) | 374 (71.1) | 393 (75.0) | 390 (74.3) |
| DKA, ^{b, c} n (%) | 1 (0.2) | 15 (2.9) | 20 (3.8) |
| Severe hypoglycemia, ^{b} n (%) | 39 (7.4) | 30 (5.7) | 23 (4.4) |
| Diarrhea, ^{d} n (%) | 27 (5.1) | 34 (6.5) | 46 (8.8) |
| Genital mycotic infection, n (%) | 15 (2.9) | 48 (9.2) | 63 (12.0) |

Unless otherwise indicated, p < 0.001;
SOTA, sotagliflozin;
PBO, placebo;
BMI, body mass index;
DKA, diabetic ketoacidosis;
LS, least square mean;
N/A, Not applicable;
SE, standard error;
SH, severe hypoglycemia;
TEAE, Treatment-emergent adverse event.
^{a}p-value of <0.001 based on Wald test.
^{b} Number of patients with ≥1 positively adjudicated event.
^{c} DKA cases were adjudicated as “yes with certainty,” “yes, probably,” “no, unlikely,” “no with certainty,” “unclassifiable,” and “insufficient data.” Positively adjudicated cases were classified as either “with certainty” or “probably.”
^{d} Discontinuation of drug due to diarrhea was: 0.4% placebo (PBO), 0.4% sotagliflozin 200 mg, and 0.8% sotagliflozin 400 mg.

Additional data from these combined studies was presented in U.S. patent application publication no. US 2024-0226125 A1, published Jul. 11, 2024, which focusses on an observed correlation between body mass index (BMI) and efficacy. In particular, it was found that subgroups distinguished by baseline BMI<27 kg/m² and baseline BMI≥27 kg/m² exhibited different efficacy profiles.

At baseline, no significant difference was observed between the high- and low-BMI groups, although overweight and obese patients with BMI≥27 kg/m² tended to have higher baseline SBP. Overall, at Week 24, the efficacy of sotagliflozin versus placebo was more pronounced in the overweight and obese subgroups with BMI≥27 kg/m², especially for the measurements of A1C, body weight, time in range, DTSQ, and 2-item DDS2. Using the same statistical models, tests of a treatment by subgroup interaction were associated with p-values<0.05 for the A1C, body weight, and DTSQ change from baseline scores. This means that for these variables, the efficacy of sotagliflozin was systematically better in patients with BMI≥27 kg/m² compared to the lower BMI subgroup. The difference tended to be greater in the 400 mg group for A1C, body weight, and time in range. The decrease in fasting plasma glucose was greater in the overweight and obese patients for the 400 mg dose only. The decrease in SBP and 2-hour postprandial glucose tended to be lower in these patients. A similar trend was observed at Week 52.

Rather than focusing on BMI, the present invention is based on discoveries centered around kidney function. In this regard, baseline characteristics (after insulin therapy optimization) of participants randomized in the pooled analysis group (in Tandem1 and in Tandem2 patients) are provided in Table 3.

TABLE 3
Baseline Characteristics of Pooled Populations

| Characteristic | Placebo (n = 76) | | 200 mg (n = 85) | | 400 mg (n = 76) | |
|---|---|---|---|---|---|---|
| Randomized, n | 526 | | 524 | | 525 | |
| Age, years (mean) | 42.5 | (13.3) | 44.4 | (13.7) | 44.0 | (13.4) |
| Females, % | 48.5 | | 49.4 | | 51.8 | |
| White race, % | 93.9 | | 94.1 | | 94.5 | |
| TID duration, years mean | 21.2 | (12.0) | 21.6 | (12.5) | 21.5 | (12.3) |
| CSII, %/MDI | 43.0/57.0 | | 42.7/57.3 | | 42.7/57.3 | |
| BMI, kg/m² | 28.5 | (5.3) | 28.9 | (5.6) | 28.7 | (5.2) |
| Weight, kg, mean | 84.3 | (17.6) | 84.5 | (18.1) | 84.2 | (18.1) |
| HbA_{1C}, % mean | 7.7 | (0.8) | 7.7 | (0.8) | 7.6 | (0.8) |
| HbA_{1C}, % mean, mmol/mol | 60.3 | (8.8) | 60.4 | (8.4) | 60.0 | (8.5) |
| Total Daily Insulin, IU/kg, mean | 0.75 | (0.3) | 0.73 | (0.3) | 0.73 | (0.3) |
| SBP, mmHg, mean | 122.0 | (14.6) | 121.5 | (15.0) | 121.3 | (14.3) |
| eGFR (MDRD), mL/min/1.73 m² | | | | | | |
| Mean | 90.2 | (18.5) | 89.3 | (19.6) | 89.1 | (18.3) |
| <60 mL/min/1.73 m² (%) | 24 | (4.6) | 22 | (4.2) | 25 | (4.8) |
| ≥60 mL/min/1.73 m² (%) | 502 | (95.4) | 502 | (95.8) | 500 | (95.2) |
| UACR, mg/g | | | | | | |
| Geometric Mean (CI) | 8.9 | (8.0, 9.8) | 9.6 | (8.7, 10.7) | 8.7 | (7.9, 9.7) |
| Median (Q1:Q3) | 6.6 | (4.3:13.0) | 7.0 | (4.4:14.1) | 6.3 | (4.2:12.3) |
| <30, n (%) | 451 | (87.7) | 439 | (85.6) | 450 | (88.4) |
| Median | 5.7 | (4.1:9.2) | 6.1 | (4.2:9.2) | 5.6 | (4.0:9.4) |
| ≥30, n (%) | 63 | (12.3) | 74 | (14.4) | 59 | (11.6) |
| Median | 56.2 | (35.7:197.2) | 61.3 | (39.9:155.1) | 83.3 | (48.0:305.0) |

Unless otherwise indicated, numbers in parentheses are standard deviations; MDRD, Modification of Diet in Renal Disease; CKD-EPI, Chronic Kidney Disease Epidemiology Collaboration; CI, confidence interval; Q, quartile.

While most patients exhibited relatively healthy kidney function, fifteen percent of patients enrolled in these studies had CKD defined as eGFR<60 mL/min/1.73 m³ and/or UACR≥30 mg/g, as shown below in Table 4.

TABLE 4
Pooled Patients By Dosage With and Without CKD

| | Placebo | | 200 mg/day | | 400 mg/day | | Total | |
|---|---|---|---|---|---|---|---|---|
| Baseline eGFR | | | | | | | | |
| (mL/min/1.73 m²) | | | | | | | | |
| Number | 526 | | 524 | | 525 | | 1575 | |
| Mean | 90.18 | | 89.30 | | 89.12 | | 89.53 | |
| Median | 89.70 | | 87.50 | | 88.00 | | 88.60 | |
| Minimum, Maximum | 43.4, 153.7 | | 46.2, 170.7 | | 40.3, 167.1 | | 40.3, 170.7 | |
| <60 mL/min/1.73 m² (%) | 24 | (4.6) | 22 | (4.2) | 25 | (4.8) | 71 | (4.5) |
| ≥60 mL/min/1.73 m² (%) | 502 | (95.4) | 502 | (95.8) | 500 | (95.2) | 1504 | (95.5) |
| Baseline Urine Albumin/ | | | | | | | | |
| Creatinine Ratio (mg/g) | | | | | | | | |
| Number | 514 | | 513 | | 509 | | 1575 | |
| Mean | 39.0874 | | 37.2136 | | 37.9464 | | 38.0834 | |
| Median | 6.5955 | | 6.9770 | | 6.2500 | | 6.5495 | |
| Minimum, Maximum | 1.829, 5227.273 | | 1.987, 2440.000 | | 1.695, 2671.875 | | 1.695, 5227.273 | |
| ≥30 mg/g (%) | 63 | (12.0) | 74 | (14.1) | 59 | (11.2) | 196 | (12.4) |
| <30 mg/g (%) | 451 | (85.7) | 439 | (83.8) | 450 | (85.7) | 1340 | (85.1) |
| Patients with CKD (%) | 76 | (14.4) | 85 | (16.2) | 76 | (14.5) | 237 | (15.0) |
| Patients without CKD (%) | 450 | (85.6) | 439 | (83.8) | 449 | (85.5) | 1338 | (85.0) |

Some baseline characteristics of the CKD subpopulation are provided below, where the values are mean values (standard deviation in parenthesis) unless otherwise noted.

TABLE 5
Baseline Characteristics of CKD Population

| Characteristic | Placebo (n = 76) | | 200 mg/day (n = 85) | | 400 mg/day (n = 76) | |
|---|---|---|---|---|---|---|
| Age (years) | 48.3 | (15.6) | 46.9 | (15.1) | 45.8 | (13.8) |
| Females, n (%) | 42 | (55) | 35 | (41) | 44 | (58) |
| TID duration, years | 26 | (12) | 27 | (12) | 24 | (13) |
| CSII, n (%) | 28 | (37) | 31 | (37) | 29 | (38) |
| BMI, kg/m² | 29.2 | (5.7) | 29.2 | (6.5) | 28.7 | (6.2) |
| HbA_{1C}, % | 7.8 | (0.9) | 7.7 | (0.8) | 7.9 | (0.8) |
| FPG, mg/dL | 169 | (77) | 165 | (78) | 150 | (63) |
| TDD Insulin, IU/kg | 0.80 | (0.47) | 0.79 | (0.48) | 0.73 | (0.27) |
| SBP, mmHg | 128 | (19) | 128 | (16) | 126 | (17) |
| eGFR, mL/min/1.73 m² | 78 | (24) | 80 | (24) | 79 | (24) |

| | | | |
|---|---|---|---|
| Median UACR, mg/g | 48.6 | 51.1 | 63.6 |

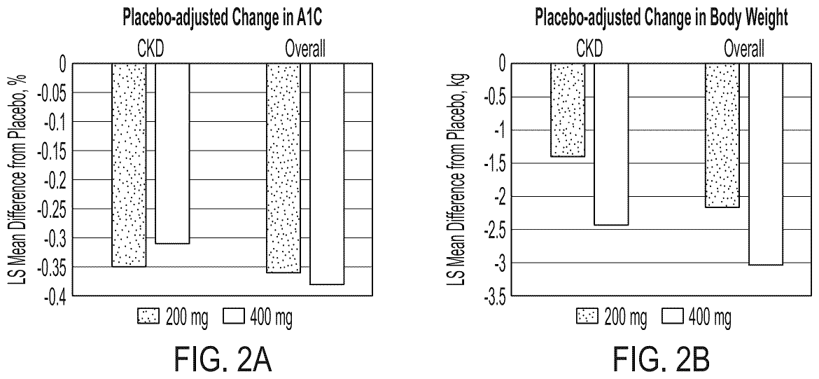

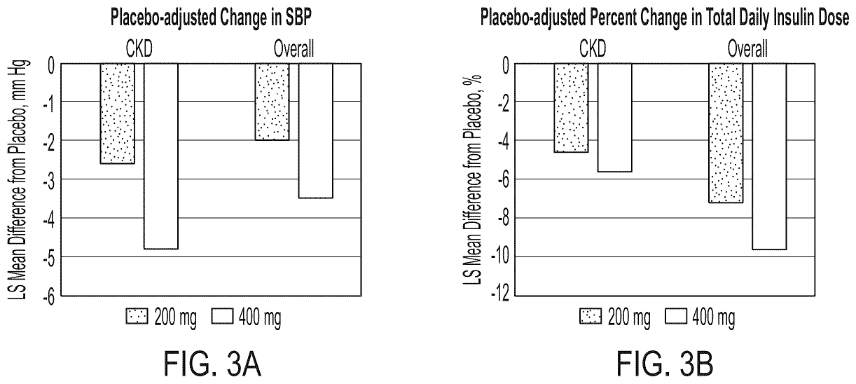

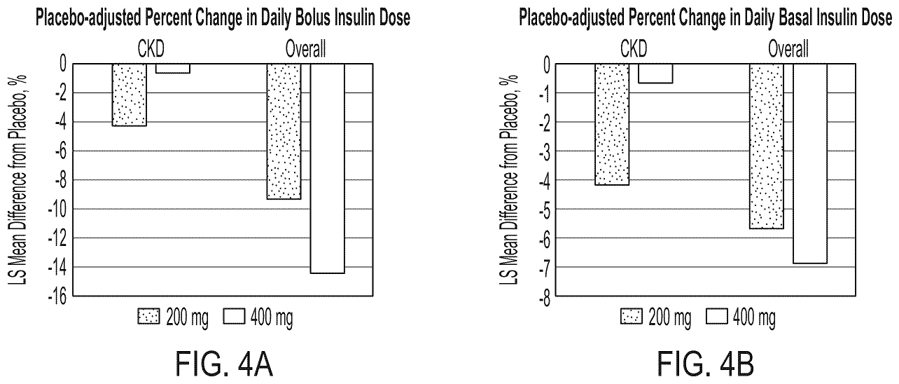

Certain characteristics of the CKD and overall (i.e., total pooled) study populations after 24 weeks of treatment with either 200 mg/day or 400 mg/day oral administration of sotagliflozin are shown in the figures. FIG. 2A shows the placebo-adjusted change in patients' HbA_{1C}, while FIG. 2B shows the placebo-adjusted change in patients' body weight. Comparisons between the populations' placebo-adjusted change in systolic blood pressure (SBP) and total daily insulin dose are shown in FIGS. 3A and 3B, respectively. Differences between the populations' placebo-adjusted change in patients' daily bolus insulin dose and daily basal insulin dose are readily apparent from FIGS. 4A and 4B, respectively.

Of particularly interest is the finding that the CKD subpopulation exhibited lower relative risk of DKA—the adverse event that to date has precluded FDA regulatory approval of sotagliflozin for the treatment of T1D—and severe hypoglycemia after 52 weeks, as shown in Table 6.

TABLE 6
Incidence of Adjudicated Severe Hypoglycemia and DKA

| Events | Placebo | 200 mg/day | 400 mg/day |
|---|---|---|---|
| Overall Cohort | N = 526 | N = 524 | N = 525 |
| Severe hypoglycemia, | 39 (7.4) | 30 (5.7) | 23 (4.4) |
| n (%) | | | |
| HR (95% CI) | — | 0.76 (0.47, 1.22) | 0.58 (0.35, 0.97) |
| DKA, n (%) | 1 (0.2) | 15 (2.9) | 20 (3.8) |
| HR (95% CI) | — | 15.0 (2.0, 113.3) | 20.3 (2.7, 151.2) |
| CKD Cohort | N = 76 | N = 85 | N = 76 |
| Severe hypoglycemia, | 13 (17.1) | 6 (7.1) | 3 (3.9) |
| n (%) | | | |
| HR (95% CI) | — | 0.34 (0.13, 0.91) | 0.2 (0.06, 0.70) |
| DKA, n (%) | 1 (1.3) | 4 (4.7) | 2 (2.6) |
| HR (95% CI) | — | 3.2 (0.4, 29.0) | 1.9 (0.2, 20.8) |

HR = Hazard Ratio, CI = confidence interval. Efficacy endpoints were presented as LS mean change from baseline at Week 24 (95% CI) to be consistent with the primary endpoint time point in the individual trials.

These data show that treatment of patients with type 1 diabetes has a better benefit risk profile with similar efficacy and improved safety in a cohort of patients with CKD relative to the overall cohort. Sotagliflozin significantly reduced A1C and BW in the CKD subgroup to a similar extent as the overall cohort, but the relative risk of severe hypoglycemia is lower with sotagliflozin compared to placebo in the CKD cohort. Similarly, the relative risk of DKA was not significantly higher with sotagliflozin relative to placebo in the CKD cohort, which contrasts with the large increase in risk in the overall cohort.

### Example 3. In Tandem3 Study in Patients with Type 1 Diabetes

The in Tandem3 study was a phase 3, double-blind trial conducted at 133 centers worldwide. In the study, 1402 patients with type 1 diabetes who were receiving treatment with insulin therapy (pump or injections) were randomly assigned to receive sotagliflozin (400 mg per day) or placebo for 24 weeks. The primary end point was a glycated hemoglobin level lower than 7.0% at week 24 with no episodes of severe hypoglycemia or diabetic ketoacidosis after randomization. Secondary end points included the change from baseline in glycated hemoglobin level, weight, systolic blood pressure, and mean daily bolus dose of insulin. Specific details of the study were reported in Garg, S. K., et al., “Effects of Sotagliflozin Added to Insulin in Patients with Type 1 Diabetes” N. Engl. J. Med. 2017:377:2337-48.

In the study, it was found that a significantly larger proportion of patients in the sotagliflozin group than in the placebo group achieved the primary end point (200 of 699 patients [28.6%] vs. 107 of 703 [15.2%], P<0.001). The least-squares mean change from baseline was significantly greater in the sotagliflozin group than in the placebo group for glycated hemoglobin (difference, −0.46 percentage points), weight (−2.98 kg), systolic blood pressure (−3.5 mm Hg), and mean daily bolus dose of insulin (−2.8 units per day) (P≤0.002 for all comparisons). The rate of severe hypoglycemia was similar in the sotagliflozin group and the placebo group (3.0% [21 patients] and 2.4% [17 patients], respectively). The rate of documented hypoglycemia with a blood glucose level of 55 mg per deciliter (3.1 mmol per liter) or below was significantly lower in the sotagliflozin group than in the placebo group. The rate of DKA was higher in the sotagliflozin group than in the placebo group (3.0% [21 patients] and 0.6%, respectively). Id. at 2337.

Some baseline characteristics of the overall and CKD patient populations are provided in Table 7:

TABLE 7
Baseline Characteristics of Total Population

| | Total Population | | | | CKD Population | | | |
|---|---|---|---|---|---|---|---|---|
| Characteristic | Placebo (n = 703) | | 400 mg (n = 699) | | Placebo (n = 113) | | 400 mg (n = 115) | |
| Age, years (sd) | 42.4 | (14.0) | 43.3 | (14.2) | 47.4 | (14.8) | 47.8 | (16.0) |
| Females, n (%) | 364 | (51.8) | 341 | (48.8) | 56 | (50) | 55 | (48) |
| TID duration, years (sd) | 19.6 | (12.1) | 20.5 | (12.4) | 23 | (13) | 24 | (14) |
| CSII, n (%) | 279 | (40) | 275 | (39) | 45 | (40) | 40 | (35) |
| BMI, kg/m² | 28.10 | (5.18) | 28.29 | (5.13) | 28.2 | (5.1) | 28.7 | (5.4) |
| HbA_{1C}, % mean | 8.21 | (0.92) | 8.26 | (0.96) | 8.3 | (1.0) | 8.7 | (1.2) |
| FPG, mg/dL | 163.4 | (69.1) | 165.1 | (71.6) | 160 | (74) | 174 | (81) |
| TDD Insulin, mean, IU/kg | 0.71 | (0.29) | 0.69 | (0.28) | 0.74 | (0.31) | 0.71 | (0.32) |
| SBP, mean mmHg | 121.8 | (14.8) | 122.0 | (15.3) | 125 | (16) | 128 | (17) |
| eGFR, mL/min/1.73m² | 92 | (23) | 92 | (20) | 79 | (27) | 82 | (25) |
| mean (SD) | | | | | | | | |

| | | | | |
|---|---|---|---|---|
| UACR, median mg/g | 7.3 | 7.4 | 61.8 | 60.2 |

CSII, continuous subcutaneous insulin infusion; FPG, fasting plasma glucose; TDD, total daily dose; SBP, systolic blood pressure

For this invention, data from CKD patients were compared to those of the overall cohort. As shown below, 16% of patients had CKD at baseline:

TABLE 8
Pooled Patients By Dosage With and Without CKD

| | Placebo | | 200 mg/day | | Total | |
|---|---|---|---|---|---|---|
| Baseline eGFR | | | | | | |
| (mL/min/1.73 m²) | | | | | | |
| Number | 703 | | 699 | | 1402 | |
| Mean | 92.47 | (21.893) | 91.51 | (19.761) | 91.99 (20.855) | |
| Median | 90.80 | | 90.40 | | 90.60 | |
| Minimum, Maximum | 41.9, 199.6 | | 43.6, 186.2 | | 41.9, 199.6 | |
| <60 mL/min/1.73 m² (%) | 42 | (6.0) | 32 | (4.6) | 74 (5.3) | |
| ≥60 mL/min/1.73 m² (%) | 661 | (94.0) | 667 | (95.4) | 1328 (94.7) | |
| Baseline Urine Albumin/ | | | | | | |
| Creatinine Ratio (mg/g) | | | | | | |
| Number | 677 | | 672 | | 1349 | |
| Mean | 49.7104 | (267.53779) | 55.5800 | (291.04136) | 52.6344 | (279.40486) |
| Median | 7.2960 | | 7.4165 | | 7.3390 | |
| Minimum, Maximum | 2.105, 5394.366 | | 1.948, 5380.282 | | 1.948, 5394.366 | |
| ≥30 mg/g (%) | 90 | (12.8) | 96 | (13.7) | 186 | (13.3) |
| <30 mg/g (%) | 587 | (83.5) | 576 | (82.4) | 1163 | (83.0) |
| Patients with CKD (%) | 113 | (16.1) | 115 | (16.5) | 228 | (16.3) |
| Patients without CKD (%) | 590 | (83.9) | 584 | (83.5) | 1174 | (83.7) |

After 24 weeks, the placebo-adjusted change in HbA1C for the CKD and overall populations were similar: a least-squared mean of −0.46 percent (standard error (SE)=0.120, p-value<0.001) was observed in the CKD group and least-squared mean of −0.46 percent (SE=0.042, p-value<0.001) in the total, overall population.

Changes in placebo-adjusted body weight were observed in both groups after 24 weeks, and the values were also similar: a least-squared mean of −2.82 kg (SE=0.464, p-value<0.001) for the CKD group and least-squared mean of −2.98 (SE=0.166, p-value<0.001) for the total, overall population.

After 16 weeks, the least-squared mean changes in the placebo-adjusted systolic blood pressures of both groups were: −7.1 mm Hg (SE=1.81, p-value<0.001) for the CKD group compared to −3.8 mm Hg (SE=0.57, p-value<0.001) for the total, overall population.

Of particular interest is the observation that the groups exhibited notable differences in their incidence of DKA after 24 weeks, as shown in Table 9.

TABLE 9
Incidence of Adjudicated Severe Hypoglycemia and DKA

| Events | Placebo | 400 mg/day | |
|---|---|---|---|
| Overall Cohort | N = 703 | N = 699 | |
| Severe hypoglycemia, n (%) | 17 (2.4) | 21 | (3.0) |
| HR, n (95% CI) | — | 1.3 | (0.7, 2.4) |
| DKA, n (%) | 4 (0.6) | 21 | (3.0) |
| HR, n (95% CI) | | 5.5 | (1.9, 15.9) |
| CKD Cohort | N = 113 | N = 115 | |
| Severe hypoglycemia, n (%) | 5 (4.4) | 8 | (7.0) |
| HR, n (95% CI) | — | 1.7 | (0.5, 5.1) |
| DKA, n (%) | 1 (0.9) | 3 | (2.6) |
| HR, n (95% CI) | | 3.5 | (0.4, 33.9) |

Although the DKA risk is numerically higher with sotagliflozin compared to placebo, the relative risk of DKA was lower in the CKD cohort (3.5) compared to the overall cohort (5.5).

In sum, analysis of results from three clinical trials shows that treatment of T1D with sotagliflozin has a better benefit/risk profile with similar efficacy and improved safety in a cohort of patients with CKD relative to the overall T1D patient population evaluated in the trials.

All publications (e.g., patents and patent applications) cited above are incorporated herein by reference in their entireties.

1-3. (canceled)

4. A method of slowing kidney function decline in a patient suffering from type 1 diabetes (T1D) while minimizing the risk of diabetic ketoacidosis (DKA), which comprises: 1) identifying a patient with T1D and chronic kidney disease (CKD); and 2) administering to the patient a therapeutically effective amount of an SGLT2 inhibitor.

5. (canceled)

6. The method of claim 4, wherein the SGLT2 inhibitor is sotagliflozin.

7. The method of claim 4, wherein the patient is on insulin therapy.

8. (canceled)

9. The method of claim 4, wherein the patient has an eGFR<60 mL/min/1.73 m².

10. The method of claim 9, wherein the patient has an eGFR<45 mL/min/1.73 m².

11. The method of claim 10, wherein the patient has an eGFR<30 mL/min/1.73 m².

12. The method of claim 4, wherein the patient has an eGFR>15 mL/min/1.73 m².

13. The method of claim 4, wherein the patient has a UACR≥30 mg/g.

14. The method of claim 13, wherein the patient has a UACR≥300 mg/g.

15. The method of claim 4, wherein the patient has an eGFR<60 mL/min/1.73 m² and a UACR≥30 mg/g.

16. The method of claim 4, wherein the patient has a BMI≥27 kg/m².

17. The method of claim 4, wherein the patient satisfies at least one of the following criteria: is an adult; is not a pregnant woman: is using either multiple daily injections or subcutaneous insulin infusion for insulin delivery; has an HbA_{1C} of 7.0-11.0%; and/or has a β-hydroxybutyrate level≤0.6 mmol/L.

18. The method of claim 17, wherein the patient is an adult.

19. The method of claim 17, wherein the patient satisfies all of the criteria.

20. The method of claim 6, wherein the sotagliflozin is administered orally.

21. The method of claim 20, wherein the sotagliflozin is administered as a tablet, caplet, or capsule.

22. (canceled)

23. (canceled)

24. The method of claim 4, which method further comprises monitoring the patient for DKA or an increased risk of DKA.

25. The method of claim 24, wherein the monitoring is done by testing the patient's blood glucose, arterial pH, and/or serum bicarbonate levels.

26. The method of claim 4, which further comprises monitoring the patient for hypoglycemia or an increased risk of hypoglycemia.

27-53. (canceled)
